(19) United States
(12) Patent Application Publication
(10) Pub. No.: US 2011/0086076 A1
(43) Pub. Date: Apr. 14, 2011

(54) COMPACT POWDER FOR SKIN CARE OR FOR MAKING UP THE SKIN

(75) Inventors: Michéle Mousset, Nogent Sur Marne (FR); Michel Siodmak, Saint Denis (FR)

(73) Assignee: CHANEL PARFUMS BEAUTE, Neuilly Sur Seine (FR)

(21) Appl. No.: 12/999,524

(22) PCT Filed: Jun. 16, 2009
(86) PCT No.: PCT/EP2009/057471

§ 371 Date: Dec. 16, 2010

(30) Foreign Application Priority Data
Jun. 17, 2008 (FR) 0853974

(51) Int. Cl. A61K 8/11; A61K 8/92; A61Q 1/02; A61Q 1/12

(52) U.S. Cl. 424/401; 424/63

(57) ABSTRACT

The subject of the present invention is a compact powder which is soft and creamy on application, and which contains a filler composed of a substrate in the form of platelets, arranged at the surface of which are particles which have an average size of between 5 nm and 10 μm and a refractive index of less than 2.

The present invention relates to a cosmetic composition for skin care or for making up the skin, which is in the form of a compact powder and comprises a particulate filler.

A large proportion of cosmetic compositions, such as blushers, eye shadows and foundations, are in the form of a powder which can be free, compacted or cast. Compact powders (or alternatively compacted powders) are constituted essentially of a mixture of coloured or uncoloured powders and of a binder comprising, in particular, fatty substances. The binder is intended to give the final product a certain density, to give the inorganic and/or organic particles of the particulate phase a certain cohesion, to give the make-up product softness and an emollient property, and to promote adhesion of said product to the skin.

These compact powders are generally applied to the skin with the fingers or using an applicator such as, for example, a sponge, a powder-puff or a brush. The user generally expects, when using such products, a comfort of application, which is reflected in particular by qualities such as softness, ability to slide and to melt on the skin, and a fine and creamy texture.

The sliding properties and the softness on application are generally associated with the nature and the amount of the selected binder. Thus, a composition should contain an amount of binder sufficient to ensure a uniform appearance thereof, to give it good spreadability during application, to prevent degradation of the makeup over time, to ensure good erodability thereof and to prevent fragmentation thereof, which may be caused in particular by impacts. Now, in some circumstances, the nature of the binder and this sufficient quantity of binder may, moreover, prove to be detrimental in terms of comfort.

Thus, oils and waxes are normally used in solid cosmetic products as binders for powders, in particular in foundation powders, eye shadows, solid mascaras, lipsticks and blushers.

By virtue of their sliding properties, oils and waxes give creamy sensations on the skin; however, when the amount of fatty substance necessary to ensure cohesion of the powders becomes large, some users can find the product tacky.

For example, it is known practice to use polybutenes as a binder, or alternatively silicone oils, as described in U.S. Pat. No. 5,023,075, EP 1 184 028, EP 792 633 and EP 1 698 323, or fluoro oils as described in application EP-A-469 602. Binders for compacted powders based on long-chain fatty acid esters, such as isocetyl stearate, are also described in U.S. Pat. No. 5,063,050 or EP 723 776.

Moreover, the production of compact powders, in practice, raises many difficulties. This is because the final composition must be sufficiently uniform and compact to be easily taken up, and to prevent, moreover, a certain fragility. Furthermore, the solid particles should be well dispersed so as to enable the application of a uniform product.

Now, to prepare the compact powders according to the present invention, a known technique can be used which consists in press-moulding the powders in the presence of the binder. In certain cases, it may be difficult to obtain a constant pressure at all points of the mould and the compact powder obtained may exhibit areas of fragility. The composition according to the invention does not have these drawbacks: it is solid, i.e. impact-resistant, and has a smooth, flat surface.

In addition, consumers look for compositions in compacted form, the smooth and creamy feel of which does not leave a feeling of residual grease on the skin after application, which is not always the case with conventional binders.

The present invention proposes to overcome the difficulties and the drawbacks of the prior art by providing a composition in the form of a compacted powder which exhibits good dispersion of the solid particles and good cohesion, which is easy to take up, optionally using a brush, and which has properties that make it comfortable to apply and to wear.

The Applicant has identified, surprisingly, that a specific composite filler constituted of a flaky substrate coated with particles of small size can result in compacted compositions with satisfactory properties, without it being necessary to add thereto an amount of binder that is usually necessary in order to obtain, in particular, good cohesion and a certain sliding property on application.

Several documents have described make-up compositions comprising composite powders having a lamellar base.

For example, the subject of document JP2002-154929 is a make-up composition comprising the combination of two powders:
A: a lamellar composite filler coated with spherical particles, and
B: a lamellar powder characterized by a minor axis/major axis ratio of between 1/1 and 1/40, a thickness/major axis ratio of less than or equal to 0.5, and a refractive index of between 1.60 and 2.15, such as boron nitride.

Document JP 2002-047138 relates to a make-up composition comprising the combination of three powders:
A: a composite filler having a lamellar base coated with spherical particles, characterized by a spherical particle/lamellar base ratio=20/80-80/20,
B: a pearlescent agent coated with a pigment, such as iron oxide-covered TiO₂-coated mica pigments,
C: a lamellar powder characterized by a minor axis/major axis ratio of between 1/1 and 1/20 and a thickness/major axis ratio of less than or equal to 0.1, such as boron nitride.

The Applicant proposes a compacted powder, the soft, creamy, smooth feel of which is comparable to that of the conventional compacted formulations, and for which the feelings of residual grease on the skin after application are reduced compared with the conventional compact powders.

The resulting compact composition is soft and creamy when taken up, and disintegrates readily on the fingers or on the brush. It is also easy to compact, has sufficient cohesion, is stable over time, is impact-resistant and has a smooth, flat surface.

According to a first embodiment, the subject of the invention is a cosmetic make-up or care composition in compacted form, comprising a particulate phase and a fatty phase, characterized in that
said particulate phase comprises at least one composite filler comprising a substrate in the form of platelets coated with organic or inorganic particles having an average size of between 5 nm and 10 μm and a refractive index of less than 2, and
in that said fatty phase represents less than 15% by weight, preferably less than 10% by weight et even less than 5% by weight, relative to the weight of the composition,
   said composite filler representing from 2% to 95% by weight relative to the total weight of the composition.

A second embodiment of this invention pertains to a cosmetic make-up or care composition in compacted form, comprising a particulate phase and a fatty phase, characterized in that
said particulate phase comprises a composite filler comprising a substrate in the form of platelets coated with inorganic particles having an average size of between 5 nm and 10 μm, chosen from alumina and aluminium hydroxide, which particles have a refractive index of less than 2, and
in that, when the fatty phase comprises one or more oils of molecular weight greater than 420 g/mol, said oil or all said oils represent(s) less than 5% by weight relative to the weight of the composition,
   said composite filler representing from 2% to 95% by weight relative to the total weight of the composition.

The compact powder according to the invention is particularly effective for reducing or eliminating skin imperfections through an optical effect, by scattering the incident light in several directions. It makes it possible in particular to unify the complexion, or to aid in fading out skin defects such as microreliefs, wrinkles, fine lines, pores or variations in skin colour. It makes it possible to visually mask skin imperfections, for example pores, marks, an uneven texture, differences in tone, or spots.

### Composite Filler

The composite filler which is part of the composition of the cosmetic products according to the invention comprises a substrate in the form of platelets coated with organic or inorganic particles, the refractive index of said particles being less than 2 and even less than 1.8. In other words, the substrate is not coated with any particles having a refractive index of 2 or more, such as TiO2, or even of 1.8 or more, such as ZnO.

The composite filler preferably represents from 8% to 95% by weight relative to the total weight of the composition, and for example from 10% to 80% by weight relative to the total weight of the composition. In one embodiment, the composition contains from 15% to 70%, or even from 40% to 60% by weight, relative to the total weight of the composition.

The average size of the composite filler can be between 0.1 and 100 microns, preferably between 5 and 50 microns, preferably less than or equal to 30 microns, 15 microns, or even 10 microns. The average size (or particle size) is defined throughout the application as the size given by the statistical particle size distribution to half the population, known as D50.

The substrate in the form of platelets is preferably characterized by three dimensions: a length, a width and a thickness, the ratio of the width to the length of the platelets preferably being between 1/1 and 1/50, and the ratio of the thickness to the length preferably being less than 1/5, preferably 1/10, and more preferably less than 1/20.

The substrate may be organic or inorganic. It is preferably chosen from lamellar powders such as inorganic silicates, talc, bismuth oxychloride, organic polymers or barium sulphate, glasses, ceramics, graphite, metal oxides, aluminas, silicas, silicates, in particular aluminosilicates and borosilicates, and synthetic mica, this list not being limiting.

The polymers that may be part of the composition are, for example, a poly (alkyl methacrylate), polyethylene naphthalate, polyalkylene terephthalate or a polyimide.

According to one preferred embodiment, the substrate is chosen from silicates, such as natural or synthetic mica. The mica may be chosen from the micas muscovite, phlogopite, tiotite, sericite, lepidolite and paragonite, synthetic micas, and mixtures thereof.

The particles are advantageously much smaller in size than the substrate, so as to cover a part of the surface of the platelets, preferably without forming a continuous layer covering said surface. Preferably, the ratio of the size of the particles to the thickness of the substrate is between 1/10 and 1/1.

Thus, the particles have an average size of between 5 nm and 10 microns, preferably between 10 and 600 nm, for example of the order of 100-200 nm or of the order of 200-300 nm. The particles arranged at the surface of the platelets preferably have an essentially identical shape, so as to confer advantageous optical effects. Their shape is also preferably uniform so as to promote light scattering, for example essentially spherical or cylindrical.

In the first embodiment of this invention, the particles are advantageously constituted of a metal oxide or hydroxide such as alumina or aluminium hydroxide or silica.

The composite filler may thus be constituted of particles of inorganic silicate, surface-coated with particles comprising alumina (Al₂O₃), aluminium hydroxide (Al(OH)₃), silica (SiO₂) or a mixture thereof. The composite filler may comprise, for example:
a mica substrate and alumina particles (INCI name: MICA & ALUMINA BEADS & DIMETHICONE or MICA & ALUMINA BEADS), or
a mica substrate and aluminium hydroxide particles (INCI name: MICA & ALUMINUM HYDROXIDE & DIMETHICONE or MICA & ALUMINUM HYDROXIDE), or
a mica substrate and silica particles (INCI name: MICA & SILICA & DIMETHICONE or MICA & SILICA).

In the second embodiment of this invention, the composite filler is constituted of particles of inorganic silicate, surface-coated with particles comprising alumina (Al₂O₃), aluminium hydroxide (Al(OH)₃) or a mixture thereof.

In the foregoing, the word “constituted” means that the composite filler does not include any other inorganic particules than those contemplated, such as TiO2 or BaSO4, for instance. However, the composite filler may advantageously comprise a silicone compound, for instance a dimethicone, preferably in an amount of between 0.1% and 5%, for example of the order of 2-3%, which may for instance used as a coating.

In both embodiments, the composite filler is preferably constituted of mica coated with alumina beads, said beads having an average size equal to 10 microns.

The proportion by mass between the substrate and the particles is preferably between 20/80 and 90/10, for example from 40/60 to 90/10, and preferably between 50/50 and 80/20, and more preferably between 70/30 and 80/20.

The composite filler used in the composition of the invention preferably has a low coefficient of friction, which can be measured, for example, with the Kinetic Friction Meter from the company CCIC. The coefficient of the composite powder measured with this machine is preferably less than 0.50, more preferably less than 0.30.

The composite filler may be purchased under the references Excel Mica JP-2® and SXI-9® from Miyoshi Kasei, or Feathery Powder 12® distributed by the company SACI-CFPA. They are characterized by a considerable soft-focus effect and by a low coefficient of friction, while at the same time being transparent.

### Other Powders

The particulate phase comprises the composite powder and preferably a second powder, said particulate phase preferably representing from 75% to 99% by weight of the total weight of the composition, more preferably from 85% to 90% by weight of the total weight of the composition.

The second powder (also referred to as filler), which is different from the composite filler described above, is chosen from inorganic and organic fillers. The term “organic fillers” is intended to mean fillers constituted of a sole organic chemical compound or fillers constituted of an organic substrate coated with another compound, which is organic or inorganic. The term “inorganic fillers” is intended to mean fillers constituted of a sole inorganic chemical compound or fillers constituted of an inorganic substrate coated with another compound, which is organic or inorganic.

Each type of filler makes it possible to provide the composition according to the invention with particular and different qualities. Thus, for example, the inorganic lamellar fillers provide softness, the inorganic spherical fillers provide good disintegration and the organic spherical fillers have a structuring role and provide softness. To obtain a composition having good cosmetic properties, it is preferable to mix at least two different fillers with complementary properties.

The inorganic filler may comprise a material chosen, for example, from talc, mica, silica, kaolin, hollow silica microspheres, glass microcapsules, titanium oxides, iron oxides and zinc oxides.

The inorganic filler may be a compound comprising TiO₂ and silica, a compound comprising mica and TiO₂, a compound comprising silica and TiO₂, or a mixture thereof. The inorganic filler may be of lamellar or non-lamellar form.

Among the inorganic fillers, mention may in particular be made of:
the elastomeric silicone powders sold by SHIN ETSU under the reference KSP,
silica-coated titanium oxide, such as Flonac TS 40 C® distributed by Eckart,
silica microbeads with a particle size of between 3 and 15 microns, such as Silica Beads SB 150® manufactured by Miyoshi, or Sunsphere H31® silica microbeads with a particle size equal to 3 microns, manufactured by Asahi Glass,
silica platelets with a particle size equal to 1.5 microns, for example Chemiceler® or Finesil F-80®, distributed by Sumitomo,
silica coated with titanium dioxide and porous silica, with a particle size, for example, equal to 0.6 micron, such as the product ACS-0050510® from Catalysts and Chemicals, such that the silica/titanium dioxide/porous silica proportion is equal to 85/5/10,
mica coated with barium sulphate and with titanium oxide, such as the product Naturaleaf® from Merck, such that the mica/barium sulphate/titanium oxide proportion is equal to 66/22/12,
a complex of silica and of cerium oxide coated with amorphous silica, with a particle size of between 1 and 10 microns, such as the product Ceriguard SC®,
a complex of silica and of titanium oxide coated with polyhydrogenomethylsiloxane, for example in proportions of 93/5/2,
platelets of sericite coated with titanium oxide, with alumina and with silica, for instance Coverleaf AR-20121 M® sold by Catalysts and Chemicals, the particle size of which is between 5 and 10 microns, and such that the sericite/titanium oxide/alumina/silica proportion is equal to 67/5/18/10,
hydrated magnesium talcs or silicates, in the form of particles with a size of generally less than 40 microns,
micas or aluminosilicates of varied compositions and which are in the form of platelets having dimensions of 2 to 200 microns, preferably 5-70 microns and a thickness of 0.1 to 5 microns, preferably of 0.2-3 microns, it being possible for these micas to be of natural origin (for example muscovite, margarite, roscoelite, lipidolite, biotite) or of synthetic origin.

The inorganic fillers may also be chosen from kaolin or aluminium silicate hydrate, boron nitrides, talcs, sericites, most titanium-micas, such as the nanotitanium-mica Coverleaf PC 2055M® from the company IKEDA, zinc oxides and titanium oxides, generally used in the form of particles having dimensions that do not exceed a few micrometres, in particular spherical titanium dioxides such as SPHERITITAN® from the company IKEDA, precipitated calcium carbonate which, in the form of particles with dimensions greater than 10 microns, magnesium carbonate, magnesium hydrogen carbonate, non-porous spherical silica, hydroxyapatite, and mixtures thereof.

The second powder may also be chosen from organic fillers, such as:
the elastomeric silicone powders sold by SHIN ETSU under the reference KSP;
tetrafluoroethylene polymer powders, such as Fluon® from the company MONTEFLUOS, or Hostaflonq® from the company HOECHST, and the lauroyllysine Aminope LL-11® from the company AJINOMOTO;
metal soaps derived from organic carboxylic acids having from 8 to 22 carbon atoms, preferably from 12 to carbon atoms, for example zinc stearate, magnesium stearate, lithium stearate, zinc laurate or magnesium myristate; these soaps have a creamy feel and facilitate the adhesion of the powder to the skin;
nonexpanded synthetic polymer powders, such as polyethylene, polyesters (for example, polyethylene isophthalate or terephthalate) and polyamides (for example, nylon), in the form of particles having dimensions of less than 50 microns, which have absorbent properties and make it possible to give the skin a velvety appearance;
powders of organic materials of natural origin, such as maize starch, wheat starch or rice starch;
microporous microspheres of acrylic polymers, such as those made of crosslinked acrylate copolymers, Polytrap® from the company DOW CORNING, and those of poly(methyl methacrylate), MICROPEARL M® or MICROPEARL M 100® from the company SEPPIC; the styrene/divinylbenzene copolymer microspheres sold under the trade name PLASTIC POWDER FPSQ® by the company TOSHIKI, which are impregnated with squalane, which is an emollient cosmetic active agent; and
the vinylidene chloride/acrylonitrile copolymer microcapsules EXPANCEL® from the company Kemanord Plast.

Powders of polyamide such as nylon, poly-beta-alamine powders, polyethylene powders, polytetrafluoroethylene powders, lauroyllysine, starch, polymeric hollow microspheres such as those made of polyvinylidene chloride/acrylonitrile, for instance Expancel®, crosslinked acrylate copolymers, for instance Polytrap®, and silicone resin microbeads, for example Tospearl®, are particularly preferred.

Preferably, the second powder has a mean particle size of less than or equal to 50 microns, preferably 30 microns, more preferably 15 microns, and preferentially less than or equal to 10 microns.

The second powder may be a powder comprising an inorganic powder and a metal soap.

In this case, the filler is preferably chosen from the composites containing 1) mica or talc, and 2) metal soaps derived from organic carboxylic acids having from 8 to 22 carbon atoms, preferably from 12 to 18 carbon atoms, for example zinc stearate, magnesium stearate, lithium stearate, zinc laurate, magnesium myristate, aluminium dimyristate, and mixtures thereof.

For example, the inorganic filler is a composite of mica and of magnesium myristate, or a composite of talc and of aluminium dimyristate, or a mixture thereof.

According to one particular embodiment, the particulate phase of the composition according to the invention comprises the combination of the composite filler, of boron nitride, of mica/magnesium myristate, and methacrylate polymer.

In this embodiment, the composition advantageously comprises 50-60% by weight of the composite powder described above, —optionally as a mixture with a composite of talc and aluminium dimyristate—, 5-15% by weight of boron nitride, 8-15% by weight of mica/magnesium myristate and 5-12% by weight of methacrylate polymer.

The fillers present in the composition are advantageously micronized before being mixed with the other ingredients.

### Pigments

The composition according to the invention may contain pigments in a proportion of 0.1% to 15% by weight relative to the total weight of the composition.

The pigments in accordance with the invention may, for example, be chosen from pigments with special effects, such as pearlescent agents, reflective pigments or interference pigments.

By way of examples of pigments with special effects, mention may be made of white pearlescent pigments, such as mica coated with titanium or with bismuth oxychloride, coloured pearlescent pigments such as mica coated with titanium and with iron oxides, mica coated with titanium and in particular with ferric blue or with chromium oxide, mica coated with titanium and with an organic pigment, and also pearlescent pigments based on bismuth oxychloride. By way of pearlescent pigments, mention may be made of the pearlescent agents Cellini® sold by Engelhard (mica-TiO₂-lake), Prestige® sold by Eckart (mica-TiO₂), and Colorona® sold by Merck (mica-TiO₂—Fe₂O₃).

Mention may also be made of pigments with an interference effect that are not fixed onto a substrate, for instance liquid crystals (Helicones HC® from Wacker), or holographic interference platelets. The pigments with special effects also comprise fluorescent pigments, whether these are substances that are fluorescent in daylight or that produce an ultraviolet fluorescence, phosphorescent pigments, photochromic pigments and thermochromic pigments.

The composition may contain reflective pigments, which may or may not be goniochromatic pigments, and may or may not be interference pigments. The reflective particles preferably have a size of at least 10 μm, for example between approximately 20 μm and approximately 50 μm.

Particles with a glass substrate coated with silver, in the form of platelets, are sold under the name MICROGLASS METASHINE REFSX 2025 PS® by the company TOYAL. Particles with a glass substrate coated with nickel/chromium/molybdenum alloy are sold under the name CRYSTAL STAR GF 550®, GF 2525® by this same company. Irrespective of their shape, the reflective particles may also be chosen from particles with a synthetic substrate coated at least partially with at least one layer of at least one metallic compound, in particular a metal oxide, chosen, for example, from titanium oxides, in particular TiO₂, iron oxides, in particular Fe₂O₃, tin oxide, chromium oxide, barium sulphate and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂ and mixtures or alloys thereof.

The reflective particles are constituted of a substrate coated with a layer of oxide. Conversely, the composite powder described above is constituted of a substrate, arranged at the surface of which are discrete particles, which may be metal oxide particles.

Thus, the reflective particles may comprise the same compounds as the composite powder, but the shape and the arrangement of these compounds are different.

By way of example of such particles, mention may, for example, be made of particles comprising a synthetic mica substrate coated with titanium dioxide, or particles of glass coated with brown iron oxide or with titanium oxide (refractive index greater than 2), with tin oxide or with a mixture thereof, for instance those sold under the trademark REFLECKS® by the company ENGELHARD. Pigments of the METASHINE 1080R® range sold by the company NIPPON SHEET GLASS CO. LTD are also suitable for the invention.

Mention may also be made of particles between 80 and 100 μm in size, comprising a synthetic mica substrate (fluorophlogopite) coated with titanium dioxide representing 12% of the total weight of the particle, sold under the name PROMINENCE® by the company NIHON KOKEN.

The composition may contain one or more goniochromatic pigments. The goniochromatic colouring agent may be chosen, for example, from multilayer interference structures and liquid-crystal colouring agents. Examples of multilayer interference structures that can be used are, for example, pigments sold under the name XIRONA by the company MERCK (Darmstadt). Mention may also be made of the INFINITE COLORS pigments from the company SHISEIDO.

The composition may also contain pigments treated with perfluoroalcohol phosphate and a silane, such as triethoxycaprylylsilane. These compounds correspond in particular to the following references:
FOTS-52 Talc JA-46R®: Talc (and) C9-15 Fluoroalcohol Phosphates (and) Triethoxy Caprylylsilane
FOTS-52 Sericite FSE®: Mica (and) C9-15 Fluoroalcohol Phosphates (and) Triethoxy Caprylylsilane
FOTS-52 Sericite GMS-4C®: Mica (and) C9-15 Fluoroalcohol Phosphates (and) Triethoxy Caprylylsilane
FOTS-52 TiO2 CR-50®: Titanium Dioxide (and) C9-15 Fluoroalcohol Phosphates (and) Triethoxy Caprylylsilane
FOTS-52 Red R-516L®: Iron Oxide (C.I. 77491) (and) C9-15 Fluoroalcohol Phosphates (and) Triethoxy Caprylylsilane
FOTS-52 Yellow LLXLO®: Iron Oxide (C.I. 77492) (and) C9-15 Fluoroalcohol Phosphates (and) Triethoxy Caprylylsilane
FOTS-52 Black BL-100®: Iron Oxide (C.I. 77499) (and) C9-15 Fluoroalcohol Phosphates (and) Triethoxy Caprylylsilane

The composition according to the invention may advantageously comprise compounds sold under the reference DERMAGLO® by the company DayGlo Color Corp.

These compounds are dyes dispersed in a resin. They correspond to the following references:
DG-R222-Polyester-3, D&C Red No. 22
DG-R228-Polyester-3, D&C Red No. 28
DG-R422-Polyester-3, D&C Red No. 22
DG-R428-Polyester-3, D&C Red No. 28
DG-11-Polyester-3, D&C Red 28 & Red 21
DG-13-Polyester-3, D&C Red 28 & Orange 5
DG-14-Polyester-3, D&C Yellow 7, Orange 5 & Red 28
DG-0205-Polyester-3, D&C Orange No. 5
DG-Y410-Polyester-3, D&C Yellow No. 10
DG-17-Polyester-3, D&C Yellow No. 7
DG-20-Polyester-3, D&C Violet No. 2.

The pigments may also be in the form of composite pigments as described in Patent Application EP 1 184 426. These composite pigments may be composed in particular of particles comprising an inorganic core, at least one binder for attaching the organic pigments to the core, and at least one organic pigment which at least partially coats the core.

### Fatty Phase

The composition may also comprise a fatty phase, which comprises at least one oil or at least one wax.

Among the oils that are part of the composition of the fatty phase, mention may be made of:
fatty acid esters, such as isopropyl myristate, isopropyl palmitate, butyl stearate, hexyl laurate, isononyl isononate, 2-ethylhexyl palmitate, 2-hexyldecyl laurate, 2-octyldecyl palmitate, 2-octyldodecyl myristate, 2-octyldodecyl lactate, 2-diethylhexyl succinate, diisostearyl malate, glyceryl triisostearate or diglyceryl triisostearate;
silicone oils, such as polymethylsiloxanes, polymethylphenylsiloxanes, polysiloxanes modified with fatty acids, fatty alcohols or polyoxyalkylenes;
castor oil or jojoba oil;
hydrocarbon oils: paraffin, squalene, petroleum jelly;
higher fatty acids, such as myristic acid, palmitic acid, stearic acid, behenic acid, oleic acid, linoleic acid, linolenic acid or isostearic acid;
higher fatty alcohols, such as cetanol, stearyl alcohol or oleyl alcohol.

Among the waxes that can be used, mention may be made of beeswaxes, lanolin waxes and China insect waxes; carnauba wax, candelilla wax, ouricury wax, cork fibre waxes, sugarcane waxes, Japan waxes, hydrogenated jojoba waxes and hydrogenated oils such as hydrogenated sunflower oil, hydrogenated castor oil, hydrogenated coconut oil and hydrogenated lanolin; microcrystalline waxes, montan waxes and ozokerites; polyethylene waxes, for example Polywax 500 or Polywax 400 sold by New Phase Technologies, waxes obtained by Fisher-Tropsch synthesis, waxy copolymers and the esters thereof, paraffin waxes, such as the paraffins S&P 206, S&P 173 and S&P 434 from Strahl & Pitsch, and silicone waxes such as polyalkoxysiloxanes and polyalkylsiloxanes.

The hydrocarbon-based waxes include long-chain linear alcohols, such as the products comprising a mixture of polyethylene and of alcohols containing 20 to 50 carbon atoms, in particular Performacol 425 or Performacol 550 (mixture in 20/80 proportions) sold by New Phase Technologies.

The composition preferably comprises less than 5%, in particular less than 4%, even better still less than 3%, less than 2%, and preferably less than 1%, by weight, of an oil of which the molecular mass is greater than 420 g/mol, or even greater than 350 g/mol or greater than 300 g/mol.

In an eye shadow or foundation composition, the proportion of the fatty phase in the composition may be between 5% and 15% by weight relative to the weight of the composition.

In a blusher or concealer composition, the proportion of the fatty phase in the composition may be between 1% and 10% by weight relative to the weight of the composition. In a face powder composition, the proportion of the fatty phase in the composition may be between 0.1% and 5% by weight relative to the weight of the composition.

In an eye shadow composition, the proportion of the fatty phase in the composition may be between 0.5 and 8% by weight relative to the weight of the composition.

### Sunscreen

The composition according to the invention advantageously contains at least one sunscreen which is active in the UV-A and/or UV-B range, chosen from the following compounds identified by their INCI name:

### Para-aminobenzoic Acid Derivatives:

PABA,
Ethyl PABA,
Ethyl Dihydroxypropyl PABA,
Ethylhexyl Dimethyl PABA sold in particular under the name “ESCALOL 507” by ISP,
Glyceryl PABA,
PEG-25 PABA sold under the name “UVINUL P25” by BASF,

### Salicylic Derivatives:

Homosalate sold under the name “EUSOLEX HMS” by RONA/EM INDUSTRIES,
Ethylhexyl Salicylate sold under the name “NEO HELIOPAN OS” by HAARMANN and REIMER,
Dipropylene glycol Salicylate sold under the name “DIPSAL” by SCHER,
TEA Salicylate, sold under the name “NEO HELIOPAN TS” by HAARMANN and REIMER,

### Dibenzoylmethane Derivatives:

Butyl Methoxydibenzoylmethane sold in particular under the trade name “PARSOL 1789” by DSM,
Isopropyl Dibenzoylmethane,

### Cinnamic Derivatives:

Ethylhexyl Methoxycinnamate sold in particular under the trade name “PARSOL MCX” by DSM,
Isopropyl Methoxy cinnamate,
Isoamyl Methoxy cinnamate sold under the trade name “NEO HELIOPAN E 1000” by HAARMANN and REIMER,
Cinoxate,
DEA Methoxycinnamate,
Diisopropyl Methylcinnamate,
Glyceryl Ethylhexanoate Dimethoxycinnamate
   β,β′-diphenylacrylate Derivatives:
Octocrylene sold in particular under the trade name “UVINUL N539” by BASF,
Etocrylene, sold in particular under the trade name “UVINUL N35” by BASF,

### Benzophenone Derivatives:

Benzophenone-1 sold under the trade name “UVINUL 400” by BASF,
Benzophenone-2 sold under the trade name “UVINUL D50” by BASF
Benzophenone-3 or Oxybenzone, sold under the trade name “UVINUL M40” by BASF,
Benzophenone-4 sold under the trade name “UVINUL MS40” by BASF,
Benzophenone-5
Benzophenone-6 sold under the trade name “HELISORB 11” by NORQUAY
Benzophenone-8 sold under the trade name “SPECTRA-SORB UV-24” by AMERICAN CYANAMID
Benzophenone-9 sold under the trade name “UVINUL DS-49” by BASF,
Benzophenone-12

### Benzylidenecamphor Derivatives:

3-Benzylidenecamphor manufactured under the name “MEXORYL SD” by CHIMEX,
4-Methylbenzylidenecamphor sold under the name “EUSOLEX 6300” by MERCK,
Benzylidenecamphor Sulfonic Acid manufactured under the name “MEXORYL SL” by CHIMEX,
Camphor Benzalkonium Methosulfate manufactured under the name “MEXORYL SO” by CHIMEX,
Terephthalylidene Dicamphor Sulfonic Acid manufactured under the name “MEXORYL SX” by CHIMEX,
Polyacrylamidomethyl Benzylidene Camphor manufactured under the name “MESORYL SW” by CHIMEX,

### Phenylbenzimidazole Derivatives:

Phenylbenzimidazole Sulfonic Acid sold in particular under the trade name “EUSOLEX 232” by MERCK,
Benzimidazilate sold under the trade name “NEO HELIOPAN AP” by HAARMANN and REIMER,

### Triazine Derivatives:

Anisotriazine sold under the trade name “TINOSORB S” by CIBA GEIGY,
Ethylhexyl triazone sold in particular under the trade name “UVINUL T150” by BASF,
Diethylhexyl Butamido Triazone sold under the trade name “UVASORB HEB” by SIGMA 3V,

### Phenylbenzotriazole Derivatives:

Drometrizole Trisiloxane sold under the name “SILATRIZOLE” by RHODIA CHIMIE,
Methylenebisbenzotriazolyltetramethylbutylphenol, sold in solid form under the trade name “MIXXIM BB/100” by FAIRMOUNT CHEMICAL, or in micronized form in an aqueous dispersion under the trade name “TINOSORB M” by CIBA SPECIALTY CHEMICALS,

### Anthranilic Derivatives:

Menthyl anthranilate sold under the trade name “NEO HELIOPAN MA” by HAARMANN and REIMER,

### Imidazoline Derivatives:

Ethylhexyl Dimethoxybenzylidene Dioxoimidazoline Propionate,

### Benzalmalonate Derivatives:

Polyorganosiloxane comprising benzalmalonate functions, sold under the trade name “PARSOL SLX” by HOFFMANN LA ROCHE
   and mixtures thereof.

The organic UV screens that are more particularly preferred are chosen from the following compounds:
Ethylhexyl Salicylate,
Butyl Methoxydibenzoylmethane,
Ethylhexyl Methoxycinnamate,
Octocrylene,
Phenylbenzimidazole Sulfonic Acid,
Terephthalylidene Dicamphor Sulfonic Acid,
Benzophenone-3,
Benzophenone-4,
Benzophenone-5,
4-Methylbenzylidenecamphor,
Benzimidazilate,
Anisotriazine,
Ethylhexyl triazone,
Diethylhexyl Butamido Triazone,
Methylenebisbenzotriazolyltetramethylbutylphenol,
Drometrizole Trisiloxane,
   and mixtures thereof.

The sunscreen is present in the composition in an amount sufficient to provide the desired protection. For example, the composition contains 5-15% by weight of Ethylhexyl Methoxycinnamate.

The composition according to the invention may also comprise any additive normally used in the cosmetics field, such as antioxidants, fragrances, preservatives or cosmetic active agents such as hydrating agents or vitamins. These additives may be present in the composition in a proportion of 0-15% by weight, relative to the total weight of the composition.

According to one embodiment, the composition of the invention is essentially anhydrous; it contains less than 5% of water added during the preparation process, present in the composition as an impurity or adsorbed by the composition. Preferably, the anhydrous composition contains less than 3% of water, or even less than 1% of water.

Of course, those skilled in the art will take care to select this or these optional additional compounds, and/or the amount thereof, in such a way that the advantageous properties of the composition according to the invention are not, or are not substantially, impaired by the addition envisaged.

The compositions according to the invention may be in the form of a cosmetic product, and especially in the form of a make-up product, in particular a foundation, a concealer product, an eye shadow, a blusher or a face powder.

The following examples illustrate the invention. In these examples, the proportions indicated are percentages by weight.

### EXAMPLE 1

A compact make-up powder having the following composition is prepared:

| INCI name/chemical | Distributor | | Amount |
|---|---|---|---|
| function | name | Trade name | as % |
| MICA & ALUMINA BEADS & | MAPRECOS SAS | SXI-9 ® | 52.5 |
| DIMETHICONE | | | |
| BORON NITRIDE | COSMO CHEM | BORON | 12.5 |
| | | NITRIDE UHP | |
| | | 1109C | |
| MICA & MAGNESIUM | KOBO PRODUCTS | MICA S/MM3 ® | 12.5 |
| MYRISTATE | INC. | | |
| METHYL METHACRYLATE | MAPRECOS SAS | TECHPOLYMER | 10.0 |
| CROSSPOLYMER | | MBP-8 ® | |
| TOCOPHEROL & ASCORBYL | EMD CHEMICALS | OXYNEX LM ® | 0.25 |
| PALMITATE & LECITHIN & | INC. | | |
| GLYCERYL STEARATE & | | | |
| GLYCERYL OLEATE & CITRIC | | | |
| ACID | | | |
| CAPRYLIC/CAPRIC | BARNET | AROLEAT | 1.0 |
| TRIGLYCERIDE & | PRODUCTS | LAVANDA ® | |
| HYDROGENATED VEGETABLE | CORPORATION | | |
| OIL & LAVANDULA STOECHAS | | | |
| JASMINUM GRANDIFLORUM | BIOLANDES | JASMINE CERA | 0.6 |
| (JASMINE) FLOWER WAX & | | ELIXIR | |
| CAPRYLIC/CAPRIC | | | |
| TRIGLYCERIDE | | | |
| AMMONIUM SILVER ZINC | CEO DELVIEL | ZEOMIC AW | 0.75 |
| ALUMINIUM SILICATE | TAIKI EUROPE | 10N ® | |
| Preservatives | | | 0.5 |
| ETHYLHEXYL | DSM | PARSOL MCX ® | 9.4 |
| METHOXYCINNAMATE | NUTRITIONAL | | |
| | PRODUCTS, | | |
| | INC. | | |

After the various constituents have been mixed, said mix is micronized by means of an airjet micronization process, using, for example, an Alpine 100AS machine sold by Hosokawa Alpine. The composition is then compacted at 36 bar.

The powder obtained can be sampled easily with a makeup brush and is not rough on the skin. It feels soft and creamy when taken up.

### EXAMPLE 2

The following composition is prepared in a manner similar to Example 1.

| | Name of | | |
|---|---|---|---|
| | distributor | | |
| INCI name/chemical | or | | % by |
| function | manufacturer | Trade name | weight |
| MICA & ALUMINA & | MAPRECOS SAS | SXI-9 ® | 11.0 |
| DIMETHICONE | | | |
| TALC & ALUMINIUM | | J13 - MT | 46.0 |
| DIMYRISTATE | | | |
| Pearlescent agents | | | 2.0 |
| Pigments | | | 2.3 |
| MAGNESIUM MYRISTATE | | SATINEX ® | 0.5 |
| MAGNESIUM STEARATE | | PALMSTAR | 0.5 |
| | | 325-BP/EP ® | |
| BORON NITRIDE | COSMO CHEM | BORON | 10.0 |
| | | NITRIDE UHP | |
| | | 1109C ® | |
| MICA & MAGNESIUM | KOBO PRODUCTS | MICA S/MM3 ® | 10.0 |
| MYRISTATE | INC. | | |
| METHYL METHACRYLATE | MAPRECOS SAS | TECHPOLYMER | 8.0 |
| CROSSPOLYMER | | MBP-8 ® | |
| Preservatives | — | — | 0.4 |
| ETHYLHEXYL | DSM | PARSOL MCX ® | 7.5 |
| METHOXYCINNAMATE | NUTRITIONAL | | |
| | PRODUCTS | | |
| | FRANCE | | |
| TOCOPHEROL & ASCORBYL | EMD CHEMICALS | OXYNEX LM ® | 0.2 |
| PALMITATE & LECITHIN & | INC. | | |
| GLYCERYL STEARATE & | | | |
| GLYCERYL OLEATE & CITRIC | | | |
| ACID | | | |
| CAPRYLIC/CAPRIC | BARNET | AROLEAT | 0.8 |
| TRIGLYCERIDE & | PRODUCTS | LAVANDA ® | |
| HYDROGENATED VEGETABLE | CORPORATION | | |
| OIL & LAVANDULA STOECHAS | CODIF | | |
| JASMINUM GRANDIFLORUM | BIOLANDES | JASMINE CERA | 0.2 |
| (JASMINE) FLOWER WAX & | | ELIXIR ® | |
| CAPRYLIC/CAPRIC | | | |
| TRIGLYCERIDE | | | |
| AMMONIUM SILVER ZINC | CEO DELVIEL | ZEOMIC AW | 0.6 |
| ALUMINIUM SILICATE | TAIKI EUROPE | 10N ® | |

The powder obtained can be sampled easily with a makeup brush and is not rough on the skin.

### EXAMPLE 3

The following composition is prepared in a manner similar to Example 1.

| INCI name/chemical | Distributor | | Amount |
|---|---|---|---|
| function | name | Trade name | as % |
| MICA & ALUMINA BEADS & | MAPRECOS SAS | SXI-9 ® | 48.8 |
| DIMETHICONE | | | |
| BORON NITRIDE | COSMO CHEM | BORON | 10.0 |
| | | NITRIDE UHP | |
| | | 1109C | |
| MICA & MAGNESIUM | KOBO PRODUCTS | MICA S/MM3 ® | 10.0 |
| MYRISTATE | INC. | | |
| METHYL METHACRYLATE | MAPRECOS SAS | TECHPOLYMER | 8.0 |
| CROSSPOLYMER | | MBP-8 ® | |
| TOCOPHEROL & ASCORBYL | EMD CHEMICALS | OXYNEX LM ® | 0.2 |
| PALMITATE & LECITHIN & | INC. | | |
| GLYCERYL STEARATE & | | | |
| GLYCERYL OLEATE & CITRIC | | | |
| ACID | | | |
| CAPRYLIC/CAPRIC | BARNET | AROLEAT | 0.8 |
| TRIGLYCERIDE & | PRODUCTS | LAVANDA ® | |
| HYDROGENATED VEGETABLE | CORPORATION | | |
| OIL & LAVANDULA STOECHAS | | | |
| JASMINUM GRANDIFLORUM | BIOLANDES | JASMINE CERA | 0.2 |
| (JASMINE) FLOWER WAX & | | ELIXIR | |
| CAPRYLIC/CAPRIC | | | |
| TRIGLYCERIDE | | | |
| AMMONIUM SILVER ZINC | CEO DELVIEL | ZEOMIC AW | 0.6 |
| ALUMINIUM SILICATE | TAIKI EUROPE | 10N ® | |
| Preservatives | | | 0.4 |
| PENTAERYTHRITYL | CRODA | CRODAMOL | 3.2 |
| TETRAISOSTEARATE | | PTIS ® | |
| Pearlescent agents | | | 8.3 |
| MAGNESIUM MYRISTATE | | SATINEX ® | 1.0 |
| MAGNESIUM STEARATE | | PALMSTAR | 1.0 |
| | | 325-BP/EP ® | |
| ETHYLHEXYL | DSM | PARSOL MCX ® | 7.5 |
| METHOXYCINNAMATE | NUTRITIONAL | | |
| | PRODUCTS, | | |
| | INC. | | |

### EXAMPLE 4

The compact make-up powders A and B, having the following compositions, are prepared in a manner similar to that described in Example 1:

| | Name of | | | |
|---|---|---|---|---|
| | distributor | | A | B |
| INCI name/chemical | or | | % by | % by |
| function | manufacture | Trade name | weight | weight |
| MICA & ALUMINA & | MAPRECOS SAS | SXI-9 ® | 10.75 | |
| DIMETHICONE | | | | |
| TALC & ALUMINIUM | | J13 - MT | 42.80 | 53.55 |
| DIMYRISTATE | | | | |
| Pearlescent agents | | | 0.5 | 0.5 |
| Pigments | | | 7.25 | 7.25 |
| MAGNESIUM MYRISTATE | | SATINEX ® | 0.5 | 0.5 |
| MAGNESIUM STEARATE | | PALMSTAR | 0.5 | 0.5 |
| | | 325-BP/EP ® | | |
| BORON NITRIDE | COSMO CHEM | BORON | 10.0 | 10.0 |
| | | NITRIDE UHP | | |
| | | 1109C ® | | |
| MICA & MAGNESIUM | KOBO PRODUCTS | MICA S/MM3 ® | 10.0 | 10.0 |
| MYRISTATE | INC. | | | |
| METHYL METHACRYLATE | MAPRECOS SAS | TECHPOLYMER | 8.0 | 8.0 |
| CROSSPOLYMER | | MBP-8 ® | | |
| Preservatives | — | — | 0.4 | 0.4 |
| ETHYLHEXYL | DSM | PARSOL MCX ® | 7.5 | 7.5 |
| METHOXYCINNAMATE | NUTRITIONAL | | | |
| | PRODUCTS | | | |
| | FRANCE | | | |
| TOCOPHEROL & | EMD CHEMICALS | OXYNEX LM ® | 0.2 | 0.2 |
| ASCORBYL PALMITATE | INC. | | | |
| & LECITHIN & | | | | |
| GLYCERYL STEARATE & | | | | |
| GLYCERYL OLEATE & | | | | |
| CITRIC ACID | | | | |
| CAPRYLIC/CAPRIC | BARNET | AROLEAT | 0.8 | 0.8 |
| TRIGLYCERIDE & | PRODUCTS | LAVANDA ® | | |
| HYDROGENATED | CORPORATION | | | |
| VEGETABLE OIL & | CODIF | | | |
| LAVANDULA STOECHAS | | | | |
| JASMINUM | BIOLANDES | JASMINE CERA | 0.2 | 0.2 |
| GRANDIFLORUM | | ELIXIR ® | | |
| (JASMINE) FLOWER | | | | |
| WAX & | | | | |
| CAPRYLIC/CAPRIC | | | | |
| TRIGLYCERIDE | | | | |
| AMMONIUM SILVER | CEO DELVIEL | ZEOMIC AW | 0.6 | 0.5 |
| ZINC ALUMINIUM | TAIKI EUROPE | 10N ® | | |
| SILICATE | | | | |

At D+1 after compacting, the following were carried out on each of compositions A and B:
a measurement of the breaking index corresponding to the number of times the composition is dropped before it breaks
a measurement of the needle penetration index,
an evaluation of the powderiness, corresponding to the ability of the product to disintegrate.

The breaking index is measured by sliding a compact having dimensions of 57 mm×45 mm×5 mm on a plastic slide which is 1.5 metres long, is at an angle of 45 degrees to the ground and ends with a wooden stop.

The compact is released from the top end of the slide. The breaking index is equal to the mean (on 8 compacts) of the number of times the compact hits the stop without breaking.

The penetration index is equal to the mean of the five measurements carried out at the centre and at the corners of a compact having dimensions of 57 mm×45 mm×5 mm, using an automatic penetrometer having the reference P734 from the company NORMALAB ANALIS. The penetration time is 5 seconds and no additional load is placed on the needle (the weight of the shaft is 47.46 g and the weight of the needle is 2.57 g).

The results are summarized in the following table:

| | Composition A | Composition B |
|---|---|---|
| Breaking index | 4 | 8 |
| Penetration index | 17.75 | 10.45 |
| Powderiness | Very good powdery | Powdery feel and |
| | feel | moderately |
| | Very good ability | satisfactory |
| | to disintegrate | disintegration |

Composition A according to the invention is less hard than composition B, and therefore exhibits an improved disintegration capacity.

1-15. (canceled)

16. Cosmetic make-up or care composition in compacted form, comprising a particulate phase and a fatty phase, wherein:
said particulate phase comprises at least one composite filler comprising a substrate in the form of platelets coated with organic or inorganic particles having an average size of between 5 nm and 10 μm and a refractive index of less than 2, and
said fatty phase represents less than 15% by weight, preferably less than 10% by weight et even less than 5% by weight, relative to the weight of the composition,
said composite filler representing from 2% to 95% by weight relative to the total weight of the composition.

17. Composition according to claim 16, wherein the particles are particles of metal oxide or hydroxide such as alumina or aluminium hydroxide or silica.

18. Cosmetic make-up or care composition in compacted form, comprising a particulate phase and a fatty phase, wherein:
said particulate phase comprises a composite filler comprising a substrate in the form of platelets coated with inorganic particles having an average size of between 5 nm and 10 μm, chosen from alumina and aluminium hydroxide, which particles have a refractive index of less than 2, and
when said fatty phase comprises one or more oils of molecular weight greater than 420 g/mol, said oil or all said oils represent(s) less than 5% by weight, preferably less than 3% by weight and more preferably less than 1% by weight, relative to the weight of the composition,
said composite filler representing from 2% to 95% by weight relative to the total weight of the composition.

19. Composition according to claim 16, wherein the substrate of the composite filler is a silicate.

20. Composition according to claim 16, wherein the silicate is natural or synthetic mica.

21. Composition according to claim 16, wherein the particles are essentially spherical or essentially cylindrical in shape.

22. Composition according to claim 16, wherein the particulate phase represents from 75% to 99% by weight relative to the total weight of the composition.

23. Composition according to claim 16, wherein it is in the form of an eye shadow, a foundation, a concealer product or a blusher.

24. Composition according to claim 16, wherein the average size of the composite powder is less than or equal to 30 microns, more preferably 15 microns, and preferentially less than or equal to 10 microns.

25. Composition according to claim 16, wherein the composite filler is chosen from the fillers having the INCI name:
MICA & ALUMINA,
MICA & ALUMINUM HYDROXIDE,
MICA & ALUMINA & DIMETHICONE
MICA & ALUMINUM HYDROXIDE & DIMETHICONE

26. Composition according to claim 16, wherein the composite filler is chosen from the fillers having the INCI name:
MICA & SILICA
MICA & SILICA & DIMETHICONE.

27. Composition according to claim 16, wherein the proportion by mass between the substrate and the particles is between 20/80 and 90/10, preferably between 70/30 and 80/20.

28. Composition according to claim 16, which also contains another powder chosen from boron nitride, crosslinked acrylate polymers, and mixtures thereof.

29. Composition according to claim 16, wherein the composite filler and the other fillers optionally present in the composition are micronized.

30. Cosmetic process for making up skin such as cheeks and eyelids, comprising topically applying thereto a composition according to claim 16.

31. Composition according to claim 18, wherein the substrate of the composite filler is a silicate.

32. Composition according to claim 18, wherein the silicate is natural or synthetic mica.

33. Composition according to claim 18, wherein the particles are essentially spherical or essentially cylindrical in shape.

34. Composition according to claim 18, wherein the particulate phase represents from 75% to 99% by weight relative to the total weight of the composition.

35. Composition according to claim 18, wherein it is in the form of an eye shadow, a foundation, a concealer product or a blusher.
